(19) United States
(12) Patent Application Publication
(10) Pub. No.: US 2026/0250644 A1
(43) Pub. Date: Aug. 27, 2026

(54) METHOD FOR PRODUCTION OF VIRUS-LIKE PARTICLES FOR GENE DELIVERY

(71) Applicant: Carnegie Mellon University, Pittsburgh, PA (US)

(72) Inventors: Yongxin ZHAO, Sewickley, PA (US); Yu Hong WANG, Pittsburgh, PA (US); Emma DiBernardo, Pittsburgh, PA (US)

(21) Appl. No.: 18/879,334

(22) PCT Filed: Jun. 29, 2023
(86) PCT No.: PCT/US2023/026571

§ 371 Date: Dec. 27, 2024

Related U.S. Application Data
(60) Provisional application No. 63/357,323, filed on Jun. 30, 2022.

(51) Int. Cl. C12N 7/02; C07K 14/025; C12N 1/20; C12N 5/071; C12N 15/10; C12N 15/70; C12N 15/85; C12R 1/19; C40B 30/00; C40B 40/02; C40B 40/06

(52) U.S. Cl. CPC C12N 7/025; C07K 14/025; C12N 1/20; C12N 5/0602; C12N 15/102; C12N 15/70; C12N 15/85; C40B 30/00; C40B 40/02; C40B 40/06; C12N 2710/22051; C12N 2800/101; C12N 2800/107; C12R 2001/19

(57) ABSTRACT

Methods for production of gene-delivery vectors suitable for animal models and humans using a bacteria/mammalian dual-expression plasmid, wherein the virus-like particles may be massively produced via heterologous expression in bacteria. Also provided are methods of increasing virus-like particle transduction efficiency and methods of directed evolution.

### CROSS REFERENCE TO RELATED APPLICATIONS

This application claims priority to and the benefit of U.S. Provisional Application No. 63/357,323, filed on Jun. 30, 2022, entitled METHOD FOR RAPID AND MASSIVE PRODUCTION OF VIRUS-LIKE PARTICLES FOR GENE DELIVERY IN MAMMALIAN CELLS, which is expressly incorporated by reference in its entirety.

### FIELD OF THE INVENTION

This disclosure relates generally to the field of gene therapy and more specifically to the production of gene-delivery vectors.

### BACKGROUND

Gene therapy is the treatment of a genetic disease by introducing genetic material to alter specific cellular function. The genetic material modifies or manipulates the expression of a gene and results in the alteration of the biological properties of living cells. Gene therapy is effective by several mechanisms, including replacing a disease-causing gene with a healthy copy of the gene, inactivating a disease-causing gene that is not properly functioning, and introducing a new or modified gene to treat a specific disease.

Virus-like particles (VLPs) are gene-delivery vectors. VLPs are viral structures that lack viral genetic material and are incapable of infection. VLPs may be produced in both mammalian and bacterial systems and are capable of both in-vivo and in-vitro assembly.

Scalable production of gene-delivery vectors like VLPs is a bottleneck. Conventional vector production processes are expensive and time consuming. Thus, it is desirable to create a scalable and cost-effective production method of gene delivery vectors for gene therapy and oncolytic virus-based therapies.

### BRIEF SUMMARY

The present disclosure addresses many of the shortcomings of conventional gene delivery vehicles and methods. The scalable and cost-effective methods and systems of the present disclosure may provide gene delivery tools suitable for animal and human models. These methods and systems may provide production of virus-like particles (VLPs) that are suitable for downstream experimental use, such as to facilitate directed evolution of the VLPs and VLPs that having customizable delivery cargo. Thus, the presently disclosed methods and systems streamline conventional production schemes that require a second plasmid or a separate packaging step.

Accordingly, the present disclosure provides a dual expression plasmid capable of producing virus-like particles in bacterial cells such as E. coli and allows selection for desirable phenotypes in mammalian cells. Exemplary desirable phenotypes include the ability to package a larger cargo, higher packaging efficiency, higher transduction efficiency and cell-type specific transduction capability. The novel plasmid system may serve as the VLPs cargo and act as a reporter for successful VLP delivery of the cargo.

The present disclosure relates to a scalable method to produce and purify virus-like particles via a bacterial and mammalian dual-expression plasmid, wherein the virus-like particles may be produced via heterologous expression in bacteria, such as Escherichia coli. The present disclosure further relates to virus-like particles comprising at least one capsid protein optimized for expression in bacterial cells and gene transfer in eukaryotic cells or cell lines.

The present disclosure also relates to a scalable production method of a gene delivery system capable of production of gene delivery tools in mammalian cells.

The present disclosure further provides a method of combining virus-like particles with liposomes to increase immune evasion and boost viral transduction efficiency.

The present disclosure further provides a dual expression system for the production of virus-like particles in bacterial cells and the selection for desirable phenotypes in mammalian cells. The system allows for scalable production of virus-like particles packaged with customized delivery cargo.

### DESCRIPTION OF THE DRAWINGS

It is to be understood that both the foregoing summary and the following drawings and detailed description may be exemplary and may not be restrictive of the aspects of the present disclosure as claimed. Certain details may be set forth in order to provide a better understanding of various features, aspects, and advantages of the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the present disclosure.

The present disclosure may be better understood by reference to the accompanying drawing sheets, in which:

FIG. 1 shows a schematic of the mass production, directed evolution, and screening of virus-like particles of the methods of the present disclosure according to certain aspects.

FIG. 2 represents a plasmid map of PEDES_JCV, wherein the JCV VP1 gene is mutated. Ara is the arabinose promoter and AMPR represents ampicillin resistance.

FIG. 3 represents a negative strain transmission electron micrograph of JCV virus-like particles of the present disclosure after purification via Capto™M Core 700.

FIG. 4A shows a confocal image of 293T cells successfully transduced with un-encapsulated JCV virus-like particles of the present disclosure taken 48 hours after transduction using 10× magnification.

FIGS. 4B & 4C illustrate 293T cells successfully transduced with un-encapsulated JCV virus-like particles of the present disclosure taken 48 hours after transduction using 20× magnification, wherein FIG. 4C is a confocal image and FIG. 4B is a brightfield image showing the nuclear localization of expressed mCherry.

FIG. 5A is a graph of transduction efficiency of virus-like particles of the present disclosure complexed with liposomes compared to un-encapsulated JCV virus-like particles, wherein efficiency is quantified by the number of cells expressing mCherry in confluent fields of view, and wherein the transduction ratio is normalized to untreated virus-like particles for each trial. The negative control included plasmid DNA treated with liposomes.

FIGS. 5B & 5C are images of HEK293F cells transduced with JCV virus-like particles of the present disclosure alone (FIG. 5B) and JCV virus-like particles of the present disclosure complexed with liposomes (FIG. 5C).

FIG. 6 shows agarose gel electrophoresis of DNA obtained from chloroform extraction and PCR of Human Embryonic Kidney (HEK) cell DNA.

FIG. 7 is a transmission electron microscopy image of JCV virus-like particles of the present disclosure purified via ultracentrifugation.

FIG. 8A is a confocal microscope image using a 10× objective and a TRITC filter set with brightfield of U2OS cells transduced with unencapsulated JCV virus-like particles of the present disclosure.

FIG. 8B is a confocal microscope image using a 10× objective and a TRITC filter of 293FT cells transduced with unencapsulated JCV virus-like particles of the present disclosure.

FIG. 9 is a graph of transduction efficiency vs volume ratio for JCV virus-like particles of the present disclosure.

### DETAILED DESCRIPTION

The present disclosure provides scalable and cost-effective methods for production of gene-delivery vectors for gene therapy and oncolytic virus-based therapies.

The present disclosure provides a method of producing virus-like particles in bacteria, the method including: mutating a DNA sequence of a gene of interest; synthesizing the mutated DNA sequence with a plasmid, wherein a generated plasmid is formed; transforming the generated plasmid into bacteria; producing a protein of interest via a method of inducing gene expression from at least one transformed bacterial cell; lysing a volume of the at least one transformed bacterial cell; separating the protein of interest from cellular debris; collecting the virus-like particles; purifying the virus-like particles, wherein the virus-like particles comprise a structure in which at least two proteins are present in an aggregated form, and wherein the at least two proteins enclose a cavity. A method of the present disclosure may further include mixing the virus-like particles with liposomes, wherein the virus-like particles are complexed with the liposomes according to the methods described herein.

The present disclosure provides methods of producing virus-like particles in bacteria, wherein mutations may be introduced into the wild-type or intermediary DNA sequences of a gene(s) of interest. The mutations may be introduced at a variable rate to enable downstream variant production and screening, including, but not limited to, introduction mutations using error-prone polymerase chain reaction (PCR). DNA shuffling is an alternative method, wherein the fragments of synthesized oligonucleotides may be rapidly melted and reannealed to achieve shuffling of the DNA sequence. While error-prone PCR and DNA shuffling have been described, other methods of introducing mutations are possible and are within the scope of the present disclosure.

The resulting mutated sequences may be synthesized by PCR. The synthesized mutated sequences may be annealed to a cognate production backbone. A method for joining the mutated sequences is Gibson Assembly®, which allows multiple DNA fragments to be joined in a single isothermal reaction via defined overlapping ends. This step rapidly joins the mutated DNA sequences with the backbone template plasmid. While the Gibson Assembly ® has been described, other methods known in the art for joining the mutated sequences are possible and are within the scope of the present disclosure.

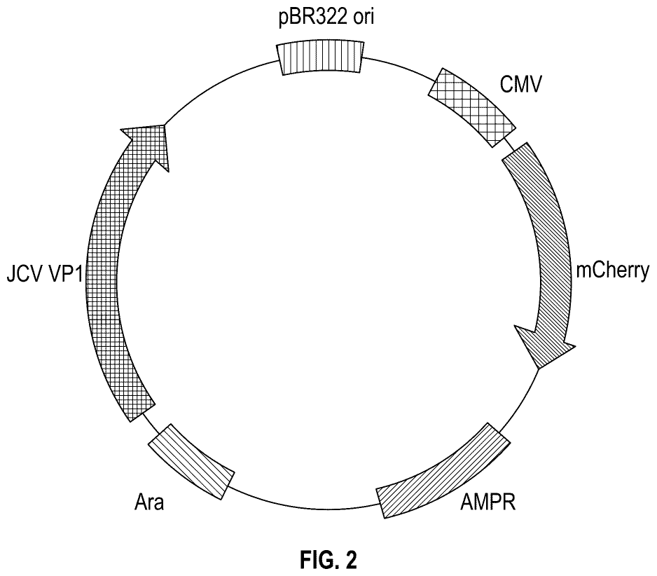

The plasmid generated from the joining of the mutated sequences may include the expression cassette for the virus-like particles (FIG. 2). The generated plasmid may be transformed into bacteria for protein production. The method of transformation may include, but is not limited to, electroporation, heat-shock transformation and chemical transformation. Bacterial cells used for transformation may be determined by one or more factors, including, but not limited to, the transformation procedure, the cell's genetic background, transformation efficiency, growth rate, and/or the like. Bacteria used for transformation may include, but is not limited to E. coli, such as E. coli strains DH5α, BL21, HB101, and JM109, or any other bacterial cell capable of taking up foreign genetic material.

Bacterial colonies that have successfully transformed the generated plasmid may be selected. The protein of interest may be produced via gene expression methods known in the art, including but not limited to, arabinose induction.

Once a sufficient volume of inducted production cells is present, the cells may be lysed in order to separate the protein of interest from the cellular debris. A virus-like particle of the present disclosure may have different properties depending on the methods of production. Thus, care must be taken to choose the method of extraction based on supernatant viscosity, ease of scalability, completeness of cell lysis, final protein concentration, and any other factors that may influence the choice of extraction.

One method of extracting and purifying the protein of interest may include the use of a multimodal resin. The present inventors have found that the dual functionality of multimodal resins, such as resins capable of size exclusion and binding chromatography, provides improved purification of the virus-like particles from the bacterial lysate. This significantly streamlines other purification protocols, such as conventional protocols that require 12 or more hours of ultracentrifugation. Use of multimodal resin with dual functionality results in streamlined extraction and purification processes of the protein. Exemplary multimodal resins of particular use in the methods of the present disclosure include, but are not limited to, Capto™ Core beads available from GE Healthcare BioSciences AB, Uppsala, Sweden.

If the supernatant has a high viscosity, the method of purification may include dialysis.

Purification is also possible with protein extraction reagents and detergents know in the art and commercially available, such as Bacterial Protein Extraction Reagent (i.e., B-PER™ available from ThermoFisher Scientific) and Cell Lytic B (i.e., CelLytic™ B, available from Sigma Aldrich®). In order to pellet the virus-like particles directly, ultracentrifugation may be used instead of concentrating the virus-like particles against a molecular weight cut-off membrane or filtration device.

The method of the present disclosure also allows for the selection of greater packaging capacity. The packaging capacity trait may be screened by increasing the size of the plasmid.

Therefore, the method of the present disclosure may produce virus-like particles capable of delivering larger cargo of at least 5 kbp DNA, or at least 6 kbp DNA, or at least 7 kbp DNA, or at least 8 kbp DNA, or at least 9 kbp DNA, or even greater, such as at least 9.4 kbp of DNA.

The method of the present disclosure may also produce virus-like particles with increased transduction efficiency through directed evolution.

The method of the present disclosure may increase transduction efficiency and shield the virus-like particles from the immune system by complexing the virus-like particles with liposomes. Mixing un-enveloped virus-like particles with liposomes overcomes two limitations of conventional virus-like particles, as it provides immune clearance by shielding virus-like particles from antibodies and increases transduction efficiency. Therefore, the methods of the present disclosure increase the viability of virus-like particles as gene-delivery vectors over conventional methods utilizing un-enveloped virus-like particles. Transduction efficiency may be quantified by the number of cells expressing a fluorescent marker, such as mCherry, a red fluorescent protein, or a green fluorescent protein.

Transduction may be completed in Human Embryonic Kidney 293FT (HEK 293FT) cell cultures. HEK 293FT cells have the SV40 large T antigen, which enables the cells to produce recombinant proteins within plasmid vectors that include the SV40 promotor. Exemplary cell cultures wherein transduction may be completed also include, but are not limited to, A549 lung cancer cells, IMR-32 neuroblastoma cells, HT-1197 bladder cancer cells, and COLO 320HSR colon cancer cells. The use of different cell lines allows for delivery optimization in different cell types through the systems and methods of the present disclosure. Therefore, transduction may be measured and optimized across various cell types such as neuronal cells over non-neuronal cells such as glial cells.

Successfully transduced cells may be measured by fluorescence and selected at the level of well plates. An alternative method of selection may include pooling the cells and separating individual cells that have been successfully transduced. This may be completed using flow cytometry and a sorting buffer.

Once the cells have been transduced with the desired phenotype, the phenotype may be tied to the genotype by extracting the nucleic acid from within the cells. The method of nucleic acid extraction may include, but is not limited to, chloroform extraction, which is applicable to both bacterial and mammalian cells, as well as proteins. While chloroform extraction is described, other methods of nucleic acid extraction are possible and within the scope of the present disclosure.

The extracted nucleic acids may then undergo PCR. Optionally, gel electrophoresis may be performed to validate the correct size of the extracted nucleic acids as well as to increase purity.

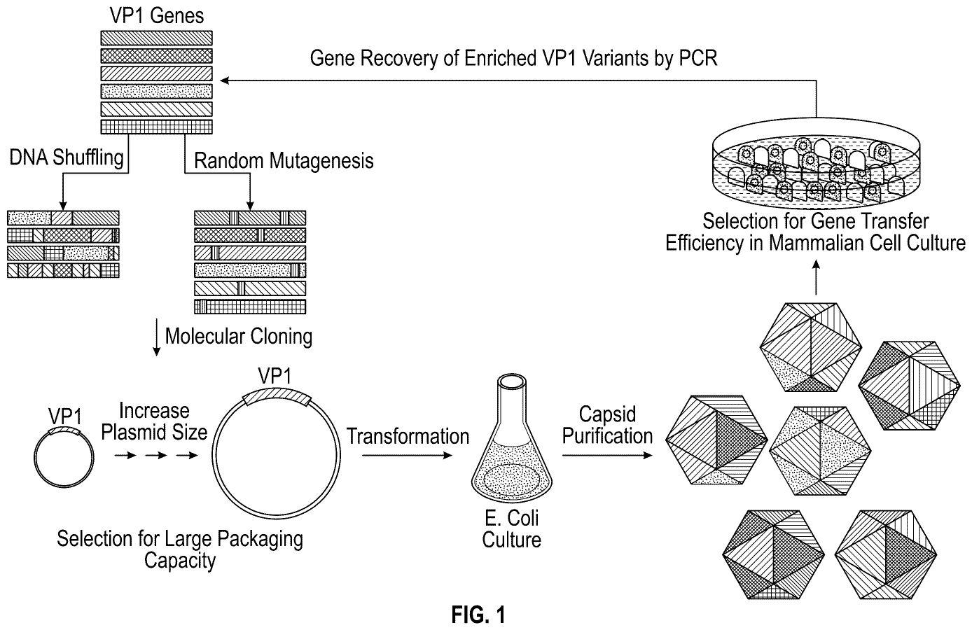

Once the nucleic acids are extracted, purified, and analyzed, the DNA may be stored at a temperature such as −20° C. The resulting DNA may be a mutated variant of the gene(s) of interest (FIG. 1). The DNA may undergo further mutagenesis by repeating the methods of the present disclosure at least a second time until a final desired phenotype and genotype is obtained.

Accordingly, the methods of the present disclosure may be performed at least 2 times, including but not limited to, 3, 4, 5, 6, 7, 8, 9, and at least 10 times.

The methods of the present disclosure allow for the production, evolution, and optimization of RNA delivery vehicles. Structural gag proteins such as Activity Regulated Cytoskeleton-associated (ARC) protein and PEG10 are capable of forming virus-like particles with diverse functions as a result of evolutionary repurposing of retroviral elements. ARC is implicated in neural processes such as synaptic plasticity, memory formation, and neurotransmitter regulation. ARC may form capsids and deliver mRNA between neurons. PEG10 is involved in mammalian placenta formation and has also been developed for mRNA delivery in mammalian cells. Structural gag proteins may be adapted to the methods of the present disclosure, as ARC and PEG10 bind to specific untranslated regions of their mRNA transcript. A construct of the gag protein virus-like particle sequence and an RNA cargo flanked by the corresponding untranslated regions may be created. The resulting capsids may be produced, optimized, and transduced in mammalian cells using the methods of the present disclosure.

The methods of the present disclosure allow for an efficient directed evolution system based on the dual expression plasmid and methods of the present disclosure. A mutant library may be expressed in bacteria and screened for successful gene delivery in mammalian cells. This system preserves mutant genotypes and allows for the recovery of mutant genes after screening. Therefore, this process may optimize virus-like particles for desired traits.

The methods of the present disclosure further allow for the large-scale production and purification of gene-delivery vectors suitable for animal models and humans. The methods of the present disclosure may be applied to any virus-like particle with the capability of self-assembly in a bacterial expression system.

### Definitions

Unless otherwise defined, all terms (including technical and scientific terms) used herein have the same meaning as commonly understood by one of ordinary skill in the art to which this disclosure belongs.

As used herein, the term “and/or” includes any and all combinations of one or more of the associated listed items. Likewise, as used in the following detailed description, the term “or” is intended to mean an inclusive “or” rather than an exclusive “or.” That is, unless specified otherwise, or clear from context, “X employs A or B” is intended to mean nay of the natural inclusive permutations. Thus, if X employs A; X employs B; or X employs both A and B, then “X employs A or B” is satisfied under any of the foregoing instances.

In the drawings, sizes, thicknesses, ratios, and dimensions of the elements may not be drawn to scale for ease of description and for clarity.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” virus-like particle, “a” gene, and “the” capsid protein may include the plural reference unless the context clearly dictates otherwise.

Words such as “then,” “next,” etc. are not intended to limit the order of the steps; these words are simply used to guide the reader through the description of the methods.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

In the description, certain details are set forth in order to provide a better understanding of various aspects of the systems and methods disclosed herein. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations.

Any minimum numerical limitation recited herein is intended to include all higher numerical limitations.

Features or functionality described with respect to certain example aspects may be combined and sub-combined in and/or with various other example aspects. Also, different aspects and/or elements of example aspects, as disclosed herein, may be combined and sub-combined in a similar manner as well. Further, some example aspects, whether individually and/or collectively, may be components of a larger system, wherein other procedures may take precedence over and/or otherwise modify their application. Additionally, a number of steps may be required before, after, and/or concurrently with example aspects, as disclosed herein. Note that any and/or all methods and/or processes, at least as disclosed herein, may be at least partially performed via at least one entity or actor in any manner.

While particular aspects have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications, and substitutions. This application including the appended claims is therefore intended to cover all such changes and modifications that may be within the scope of this application.

The term “virus-like particle (VLP)” may be understood to be a particulate structure in which a plurality of proteins is present in aggregated form, wherein they preferably enclose a cavity. At least a part of the structure-forming proteins is identical to, or derived from, viral structural proteins (capsid proteins), such as from viruses of the Papoviridae family, which includes both the Papillomaviridae and Polyomaviridae. The VLPs, however, may also originate from other virus families such as, e.g., the Parvoviridae, Flavoviridae and Retroviridae families.

Preferably, a VLP is formed by 60, 72, 120, 180, 240, 300, 360 and more than 360 viral structural proteins and may have a tubular or spherical structure. A VLP made of 360 structural proteins is usually made up of 72 pentamers which are each formed by five monomeric structural proteins. The aggregation of the structural proteins and pentamers may proceed via non-covalent or covalent bonding of the proteins. In the case of a covalent bonding, a formation of disulfide bridges is preferred.

A VLP may be composed either of a multiplicity of only one structural protein, or else of different structural proteins. Preference is given to the presence of only one structural protein, namely VP1.

The structural proteins of the VLPs, in particular VP1 (or else VP2 and/or VP3), may be identical to or derived from the structural proteins, e.g. of the following viruses from the Polyomaviridae family: African green monkey polyomavirus (AGMPy V), pavian polyomavirus 2 (BPy V-2), human polyomavirus 1 (BK virus, BKV or BKPyV), human polyomavirus 2 (John Cunningham virus, JC virus, JCV or JCPy V), bovine polyomavirus (BPyV), budgerigar polyomavirus (polyomavirus of budgerigar fledging disease, BFPy V), hamster polyomavirus (HaPyV), murine pneumotropic virus (MPtV), murine polyomavirus (MPy V), rabbit polyomavirus (rabbit kidney vacuolating virus, RKV), simian virus 12 (SV-12), simian virus 40 (SV-40), crow polyomavirus, goose hemorrhagic polyomavirus (GHPV), merkel cell polyomavirus, chimpanzee polyomavirus, finch polyomavirus and KI polyomavirus (KIV).

However, the VLPs may also correspond to the structural proteins of the self-assembling viruses. For example, the VLPs may correspond to structural proteins such as human retroviral structural gag-like proteins. According to certain aspects, the structural proteins may preferably correspond to the L1, (and also L2), of the Papillomaviridae family, or are derived therefrom, namely, for example, from the following virus genera: Alphapapillomavirus, Betapapillomavirus, Gam mapapillomavirus, Deltapapillomavirus, Epsilonpapillomavirus, Zetapapillomavirus, Etapapillomavirus, Thetapapillomavirus, lotapapillomavirus, Kappapapillomavirus, Lam bdapapillomavirus, Mupapillomavirus, Nupapillomavirus, Xipapillomavirus, Om ikronpapillomavirus, Pipapillomavirus, Trichosurus-vulpecula-Papillomavirus, and Opossum-Papillomavirus.

The VLP may, in addition, have one or more additional heterologous proteins in the capsid, i.e., proteins that are not identical or similar to a protein of a virus of the Papoviridae family. Suitable heterologous proteins are in principle all proteins which may be incorporated into the capsid, or bind to the capsid, and do not significantly impair the assembly of the VLP.

A “pentamer” in the context of the invention is a structure which is formed by five polypeptide subunits. The bonding between the individual polypeptide subunits may proceed via noncovalent or covalent bonding. The five subunits frequently form a ring-shaped structure having pentagonal symmetry. Here, generally, each subunit interacts with two adjacent subunits in each case.

“Chromatography” denotes a method which permits the separation of a mixture of substances by differing distribution of the individual components thereof between a stationary phase and a mobile phase. Centrifugation in this sense is not chromatography.

The terms “express” and “produce” are used synonymously herein and refer to the biosynthesis of a gene product. These terms encompass the transcription of a gene into RNA. These terms also encompass translation of RNA into one or more polypeptides, and further encompass all naturally occurring post-transcriptional and post-translational modifications.

“Polynucleotide,” synonymously referred to as “nucleic acid molecule.” “nucleotides” or “nucleic acids,” refers to any polyribonucleotide or polydeoxyribonucleotide, which may be unmodified RNA or DNA or modified RNA or DNA. “Polynucleotides” include, without limitation single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

In the description, certain details are set forth in order to provide a better understanding of various aspects of the methods disclosed herein. However, one skilled in the art will understand that these aspects may be practiced without these details and/or in the absence of any details not described herein. In other instances, well-known structures, methods, and/or techniques associated with methods of practicing the various aspects may not be shown or described in detail to avoid unnecessarily obscuring descriptions of other details of the various aspects.

### Aspects

Aspect 1: A method of producing virus-like particles in bacteria comprising: mutating a DNA sequence of a gene of interest; synthesizing the mutated DNA sequence with a plasmid, wherein a generated plasmid is formed; transforming the generated plasmid into bacteria; producing a protein of interest via a method of inducing gene expression from at least one transformed bacterial cell; lysing a volume of the at least one transformed bacterial cell; separating the protein of interest from cellular debris; collecting the virus-like particles; purifying the virus-like particles, wherein the virus-like particles comprise a structure in which at least two proteins are present in an aggregated form, and wherein the at least two proteins enclose a cavity; and mixing the virus-like particles with liposomes, wherein the virus-like particles are complexed with the liposomes.

Aspect 2: The method according to aspect 1, wherein the complexed virus-like particles have a higher transduction efficiency compared to un-enveloped virus-like particles.

Aspect 3: The method according to any of the foregoing aspects, wherein the plasmid size is increased, and wherein the virus-like particles have an increased packaging capacity of at least 5 kbp DNA.

Aspect 4: The method according to any of the foregoing aspects, wherein the plasmid size is increased, and wherein the virus-like particles have an increased packaging capacity of at least 7 kbp DNA.

Aspect 5: The method according to any of the foregoing aspects, wherein the plasmid size is increased, and wherein the virus-like particles have an increased packaging capacity of at least 9.4 kbp DNA.

Aspect 6: The method according to any of the foregoing aspects, wherein purifying the virus-like particles comprises using multimodal resin.

Aspect 7: The method according to any of the foregoing aspects, wherein the method is repeated at least a second time until a selected virus-like particle phenotype(s) is obtained.

Aspect 8: The method according to any of the foregoing aspects, wherein the method is repeated to select for a phenotype(s).

Aspect 9: The method according to any of the foregoing aspects, wherein the method of inducing gene expression from at least one transformed bacterial cell comprises arabinose induction.

Aspect 10: The method according to any of the foregoing aspects, wherein the bacteria is Escherichia coli.

Aspect 11: The method according to any of the foregoing aspects, further comprising a method of directed evolution, wherein the method of directed evolution comprises expressing a mutant DNA library in bacteria; screening for successful gene delivery; and recovering mutant genes.

Aspect 12: A dual expression plasmid, wherein the dual expression plasmid is capable of producing at least one virus-like particle, and wherein the dual expression plasmid is selected for at least one phenotype in a mammalian cell.

Aspect 13: The dual expression plasmid according to any of the foregoing aspects, wherein the dual expression plasmid is transformed into Escherichia coli.

Aspect 14: The dual expression plasmid according to any of the foregoing aspects, wherein the dual expression plasmid comprises an expression cassette for major capsid protein 1 (VP1) of the Jon Cunningham Virus (JCV or human polyomavirus 2).

Aspect 15: The dual expression plasmid according to any of the foregoing aspects, wherein the plasmid produces a virus-like particle having a packaging capacity of at least 5 kbp DNA.

Aspect 16: A method of producing virus-like particles in bacteria comprising: mutating a DNA sequence of a gene of interest; synthesizing a mutated sequence with a plasmid, wherein a generated plasmid is formed; transforming the generated plasmid into a bacteria cell; producing a protein of interest via a method of inducing gene expression from at least one transformed bacterial cell; lysing a volume of the at least one transformed bacterial cell; separating the protein of interest from cellular debris; collecting the virus-like particles; and purifying the virus-like particles, wherein the virus-like particles comprise a structure in which at least two proteins are present in an aggregated form, and wherein the at least two proteins enclose a cavity.

Aspect 17: The method according to any of the foregoing aspects, wherein the plasmid size is increased, and wherein the virus-like particles have an increased packaging capacity of at least 5 kbp DNA.

Aspect 18: The method according to any of the foregoing aspects, wherein the plasmid size is increased, and wherein the virus-like particles have an increased packaging capacity of at least 7 kbp DNA.

Aspect 19: The method according to any of the foregoing aspects, wherein the plasmid size is increased, and wherein the virus-like particles have an increased packaging capacity of at least 9.4 kbp DNA.

Aspect 20: The method according to any of the foregoing aspects, wherein purifying the virus-like particles comprises using multimodal resin.

Aspect 21: The method according to any of the foregoing aspects, wherein the method is repeated at least a second time until a selected virus-like particle phenotype(s) is obtained.

Aspect 22: The method according to any of the foregoing aspects, wherein the method is repeated to select for a trait(s).

Aspect 23: The method according to any of the foregoing aspects, wherein the method of inducing gene expression from at least one transformed bacterial cell comprises arabinose induction.

Aspect 24: The method according to any of the foregoing aspects, further comprising a method of directed evolution, wherein the method of directed evolution comprises expressing a mutant DNA library in bacteria; screening for successful gene delivery; and recovering mutant genes.

Aspect 25: The method according to any of the foregoing aspects, wherein the transduction efficiency of the virus-like particle is increased by a method comprising: transducing a virus-like particle in a mammalian cell; selecting the mammalian cell with a selected phenotype; extracting at least one nucleic acid from the selected mammalian cell; and purifying the at least one nucleic acid.

Aspect 26: The method according to any of the foregoing aspects, wherein the at least one nucleic acid is extracted by chloroform extraction.

Aspect 27: The method according to any of the foregoing aspects, further comprising optionally validating a size and a purity of the virus-like particle by gel electrophoresis.

Aspect 28: The method according to any of the foregoing aspects, wherein the at least one purified nucleic acid undergoes a round of mutagenesis until a selected phenotype and/or genotype is obtained.

### EXAMPLES

Specific exemplary protocols for the various steps in the methods disclosed herein are provided below. These examples are provided in substantially sequential order for the methods of directed evolution, as illustrated in FIG. 1 and discussed hereinabove, and encompass exemplary protocols to execute the methods of the present disclosure, from the mutagenesis of the wild type sequences to their manufacture in the production cells, their transduction, complexation and screening, and finally, the extraction of the nucleic acid contained within the VLPs and transduced cells. The extracted sequence is subjected to this experimental cycle again, beginning with mutagenesis.

### Example 1A

### Introduction of Mutations—Mutagenesis Via Error-prone PCR

Mutations were introduced into the wild-type of an intermediary sequence at a variable rate for downstream variant production and screening.

Reagents included: mutagenesis forward and backwards Gibson primers; Taq DNA polymerase and 10x buffer; Magnesium Chloride (7 mM); Manganese Chloride (25 mM); Deoxynucleoside triphosphates (dNTPs) (10 mM of each nucleotide); and Deionized Water.

Error-prone PCY was set up in a 50 μL reaction, wherein MnCl₂ and MgCl₂ were added to decrease nucleotide fidelity (about 2.5 mM and 7 mM final concentration, respectively). dNTPs were not added in the same concentrations to further encourage mutagenesis (ex. 1 mM dATP and dGTP, 200 μM dCTP and dTTP). Water was added to achieve 50 μLs, and Taq polymerase was added last. PCR was performed for 18 to 20 cycles, wherein the number of cycles was increased or decreased to achieve the target number of mutations. Extension time was increased to 2 minutes.

### Example 1A

### Introduction of Mutations—DNA Shuffling

An alternative to error prone PCR includes DNA shuffling, wherein fragments of synthesized oligonucleotides are rapidly melted and reannealed to achieve shuffling of the sequence.

Reagents included: mutagenesis forward and backwards Gibson primers; Taq DNA polymerase and 10× buffer; Magnesium Chloride (7 mM); Manganese Chloride (25 mM);

Deoxynucleoside triphosphates (dNTPs) (10 mM of each nucleotide); and Deionized Water.

PCR was performed as in the error-prone PCR protocol above except with changed heating conditions, wherein cycle number was increased while annealing and extension time was greatly reduced. Less mutagenic conditions were used: MgCl₂ and dNTP levels were altered to reduce mutagenic conditions, no MnCl₂.

### Example 2

### Forming Mutant Constructs-Gibson Assembly® of Mutant Constructs

After the mutated sequences were synthesized by PCR, they were annealed to the cognate production backbone via Gibson Assembly.

Reagents included: Forward and backwards Gibson backbone primers; Backbone template plasmid; Mutagenized sequences with cognate Gibson overlaps; Thermofisher Hi-Fi Gibson master mix; and Deionized Water. Equipment included: Thermocycler, Nanodrop, and Plate Reader.

Gibson primers were used to PCR out the backbone. Restriction enzyme digest with Dpnl was used after PCR to degrade the original template. The backbone band size was confirmed by gel electrophoresis.

The backbone was collected, and a concentration was inserted via nanodrop. The amount of insert and backbone to incubate with Gibson master mix was calculated, as Hi-Fi Gibson master mix for a 1:1 reaction required 0.08 pmol of each fragment.

The Gibson reaction was performed, wherein 10 μL of master mix was added to the fragments, and the total volume was increased to 20 μL, the solution was incubated at 50° C. for 30 minutes and placed on ice.

### Example 3

### Introduction of Mutant Constructs Into Bacterial Cells—Electroporation of Gibson Assembled Plasmids into Bacterial Cells

The generated plasmid containing the expression cassette for the VLPs was transformed into E. coli for protein production.

Reagents included: Electro-competent DH 10B E. coli; Super Optimal broth with Catabolite repression (SOC) media; LB growth media; Gibson assembled product; Petri Dishes with Luria Broth (LB) agar and ampicillin; Electroporation cuvettes; and Ice bath.

The cuvettes, Gibson product, and cells were incubated on ice. 1 μL of Gibson reaction was mixed with about 30 μL of competent cells in cuvettes. The electroporator was set to 2.0 kV and the cuvette was pulsed. The measured kV and time constant were recorded. 500 μL of SOC medium was immediately added into the cuvette. The contents of cuvette were transferred into 2 mL microcentrifuge tubes, which were left for outgrowth at 37° C. shaking for 1 hour. About 200 μL of the outgrowth media was transferred onto LB-ampicillin petri dishes. The petri dishes were grown overnight in a 37° C. incubator.

### Example 4

### Protein Production—Bacterial Production of VLPs

Bacterial colonies that have successfully transformed the Gibson product were selected and the protein of interest was produced via arabinose induction.

Reagents included: Plate containing grown E. coli colonies; 10% L-arabinose solution; Ampicillin (1000×); Luria Broth (LB) growth media; and TB growth media.

Culture flasks with 3 mL LB media were prepared and 3 μL ampicillin solution were added. Individual colonies on the petri dish were selected and deposited into the culture flasks. They were left to grow overnight in a 37° C. incubator, shaking. The next day, about 1 mL of the growth media was transferred into 100 mL of TB media in a baffled flask and left to grow for about 4 hours at 37° C., shaking. The solution was induced with 0.03% (final concentration) arabinose. The solution was grown overnight in 37° C., shaking.

### Example 5

### Protein Extraction

Cells were lysed and the protein of interest was separated from the cell debris.

Reagents included: Induced TB flasks; Hanks-buffered salt solution (HBSS) with calcium; Lysozyme; Halt protease inhibitor; and Benzonase.

The contents of induced TB flasks were transferred into centrifuge tubes. The tubes were spun at 5000 g for 15 minutes to pellet the cells, and the supernatant was aspirated. The proteins were extracted from the pellet using either lysozyme or commercial extraction buffers.

### Example 5A

### Lysozyme Extraction

5 mL of HBSS, 10 μL of lysozyme, and 100 μL of Halt protease inhibitor was added per 100 mL of original bacterial culture. The solution was freeze-thawed in liquid nitrogen 5 times. 1 μL Benzonase was added, and the solution was incubated at 37° C. for 2 hours.

### Example 5B

### Extraction Using B-PER™ Bacterial Protein Extraction Buffer or CelLytic™ B Lysis Reagent

The pellet from the centrifuged tubes was weighed. 5 mL of Bacterial Protein Extraction Reagent (B-PER™) or CelLytic™ B were added per gram of pellet. Note that Lysozyme and Benzonase may be added here but are not necessary. The solution was spun down at 4° C. at 10,000 g, and the supernatant was aspirated.

### Example 6

### Protein Purification

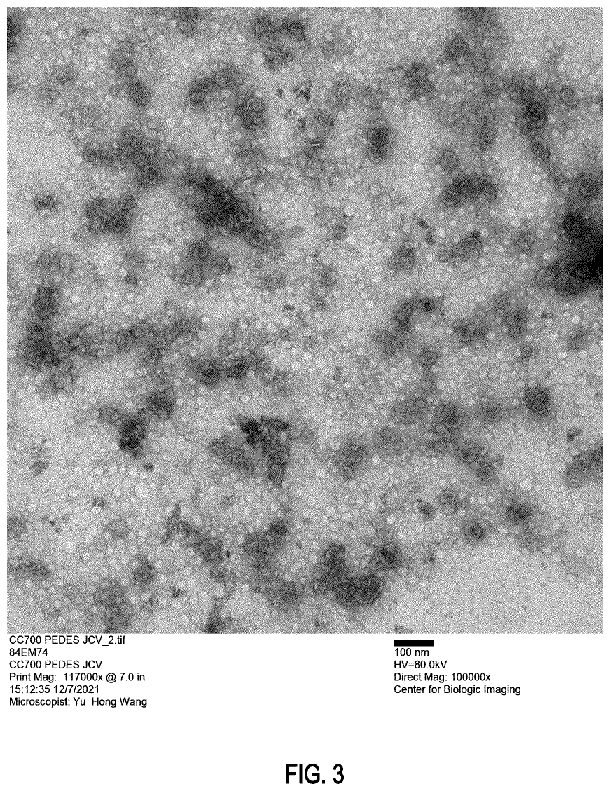

The extracted proteins were purified using the methods disclosed in Examples 6A through 6C. An exemplary product produced via the methods of the present disclosure is shown in FIG. 3, which illustrates a negative stain transmission micrograph of JCV VLPs after purification via the methods of Example 6A, i.e., purification using multimodal chromatography.

### Example 6A

### Protein Purification Via Multimodal Chromatography

Reagents included: Hanks-buffered salt solution (HBSS) with calcium; High Screen Capto™ Core 400 and 700; and Sigma 100MWCO protein concentrators.

The supernatant was transferred from the extraction protocol shown in Example 5 to 100 μL Capto™ Core 400 or 700, which was inverted at room temperature for 45 minutes, then centrifuged at 800 g for 10 minutes at room temperature, and then the supernatant was transferred to a new tube and the Capto™ Core process was repeated again. This process was repeated 1 to 2 times. The supernatant was transferred into a protein concentrator and spun at 10,000 g for 1-2 hours. The retentate was resuspended and concentrated in the protein concentrator with 200 μL of HBSS and 30 μL was aliquoted into microcentrifuge tubes and stored at −80° C.

### Example 6B

### Protein Purification via Dialysis

This example provides an alternative means for protein purification if the freeze-thawed supernatant is too viscous for the protein concentrator.

The supernatant was transferred from the extraction protocol shown in Example 5 to 30MWCO dialysis tubing and incubated in HBSS transfer buffer. The supernatant was transferred into the protein concentrator and spun at 10,000 g for 1-2 hours. The retentate was resuspended and concentrated in the protein concentrator with 200 μL of HBSS and 30 μL was aliquoted into microcentrifuge tubes and stored at −80° C.

### Example 6C

### Protein Purification via Ultracentrifugation

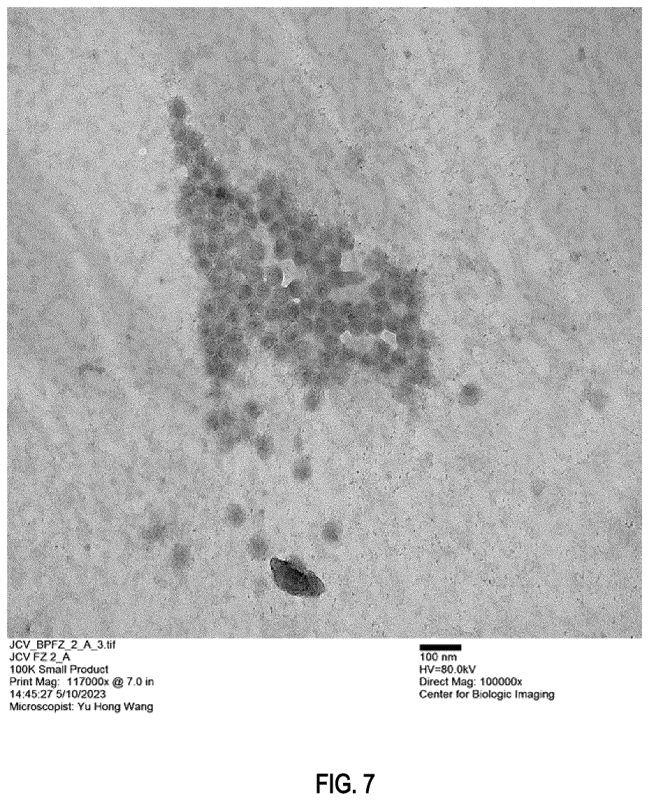

A series of ultracentrifugation and clarification spins were used to concentrate the VLPs. Endotoxin contaminants from bacterial cell lysis were removed via Triton X-114, which was subsequently removed from the final purified sample with absorbent Bio-beads. The ultracentrifugation and clarification spins resulted in less contaminated virus-like particles compared to other methods known in the art (FIG. 7). The method was adapted from Kreitz et. al, Programmable protein delivery with a bacterial contractile injection system. 2023. Nature.

Reagents included: VLPs extracted according to the methods of the present disclosure; HBSS; Triton X-114 (Thermo); Bio-beads SM-2 (Bio Rad).

Cold HBSS was added to extracted lysate containing the VLPs in an ultracentrifuge tube. The tube was filled to the recommended volume. The tube was spun at 120,000 g for 2 hours to pellet the virus-like particles. The pellet was resuspended with 1 mL HBSS and shaken at 4° C. for 30 minutes. The dissolved pellet was clarified by spinning at 16,000 g for 15 minutes.

The supernatant containing the protein was collected and the previous steps were repeated another two times. The final sample was diluted into 1 mL cold HBSS and 20 μL Triton X-114.

For a detergent wash, the sample was incubated at 4° C. in a tube turner for 30 minutes and transferred to a 37° C. heat block for 10 minutes to precipitate the detergent. The sample was spun at 20,000 g for 20 minutes at 37° C. to separate the protein and detergent phases. The protein was extracted from the upper phase and the procedure was repeated 2 more times.

For detergent removal and sample storage, the final protein phase was incubated with 300 mg Bio-Beads SM-2 in a tube turner overnight at 4° C. The sample was left to settle at room temperature for 30 minutes. The supernatant was extracted and filtered through a 0.22 μm filter. The samples were stored at 4° C. for at most a week and at −80° C. for longer.

FIG. 7 is a transmission electron microscopy (TEM) image of JCV virus-like particles of the present disclosure purified via the method described herein. The particles were uniform in size (about 40-50 nm) and exhibited capsid structure.

### Example 7A

### Mammalian Cell Transduction, Liposome Encapsulation, and Screening

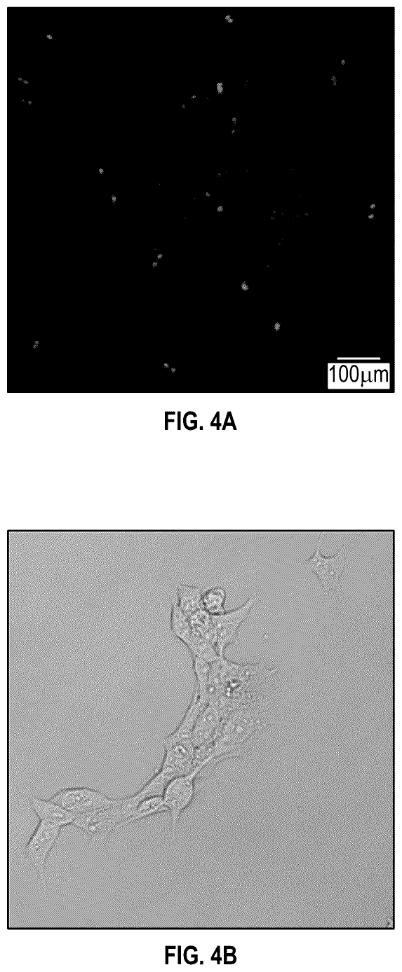

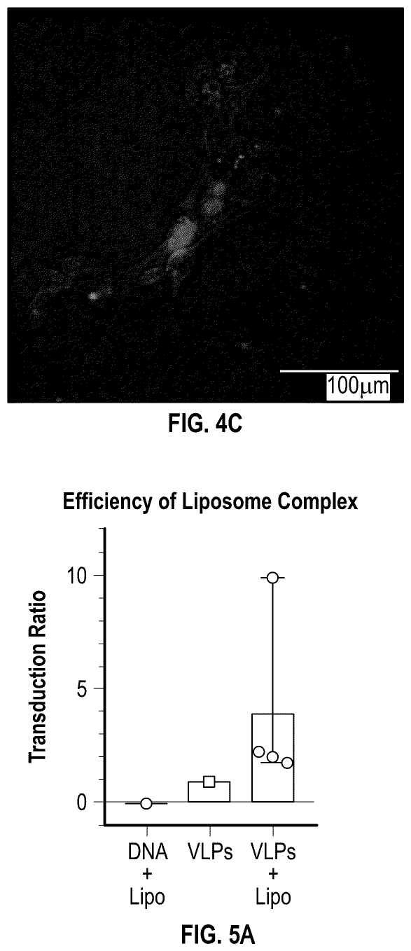

Producing VLPs in bacteria allows for selection of greater packaging capacity, which may be screened by increasing the plasmid size. Another goal of directed evolution is increasing the VLP's transduction efficiency. This was tested in HEK cell cultures. An example of successful transduction is shown in FIG. 4A-4C, wherein FIG. 4A shows a confocal image of HEK 293T cells successfully transduced with un-encapsulated JCV virus-like particles taken 48 hours after transduction using 10× magnification. FIGS. 4B and 4C illustrate HEK 293FT cells successfully transduced with un-encapsulated JCV virus-like particles taken 48 hours after transduction using 20× magnification, wherein FIG. 4C is a confocal image and FIG. 4B is a brightfield image showing the nuclear localization of expressed mCherry.

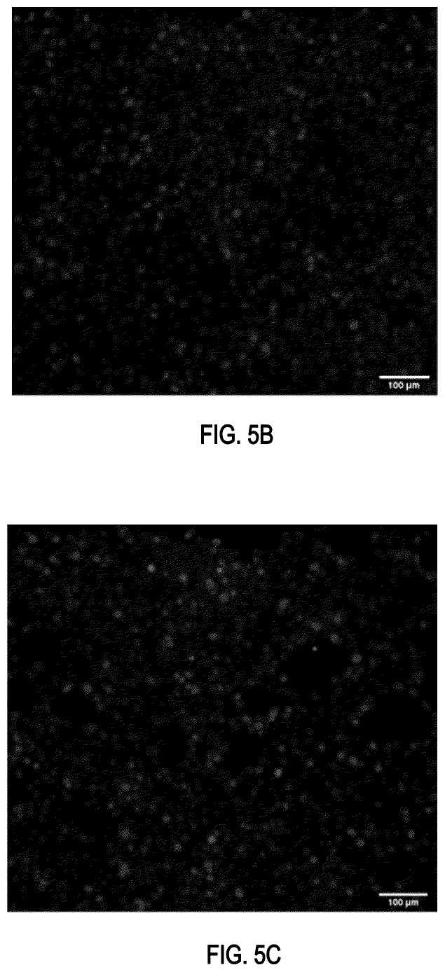

Methods that increase transduction efficiency and shield the VLPs from the immune system by complexing the particles with liposomes are also provided. This increased efficiency is quantified and shown in FIG. 5A, which shows a graph of transduction efficiency of virus-like particles encapsulated in liposomes compared to un-encapsulated JCV virus-like particles. Efficiency was quantified by the number of cells expressing mCherry in confluent fields of view, wherein the transduction ratio was normalized to untreated virus-like particles for each trial. The negative control included plasmid DNA treated with liposomes. FIGS. 5B & 5C are images of HEK293F cells transduced with JCV virus-like particles of the present disclosure alone (FIG. 5B) and JCV virus-like particles of the present disclosure complexed with liposomes (FIG. 5C). The JCV virus-like particles of the present disclosure alone resulted in 7.12 expressing cells per 5000 cells. The JCV virus-like particles of the present disclosure complexed with liposomes resulted in 14.87 expressing cells per 5000 cells.

Reagents included: Purified VLPs; 293FT cells; Dulbecco's Modified Eagle Medium (DMEM), high glucose, pyruvate, 1-glutamine, non-essential amino acids; Fetal Bovine Serum (FBS); Trypsin; Geneticin 100×; DNAsel; 1× PBS; Altogen Biosystems HEK293 Transfection Reagent Well plates were seeded with HEK cells, which were grown in 5% FBS complete media in a 37° C. incubator under 5% CO₂ until 50% confluence (at least 24 hours). The virus-like particles were sterile filter purified with a 0.2 um filter and then treated with DNAsel and incubated for 30 minutes at 25° C. Up to 1/20 of final media volume of purified virus-like particles was added to the wells along with 5 μL of DNAsel. The wells were left to transduce at 37° C. for 48 hours.

For liposome coated virus-like particles: Purified, filtered and DNAsel treated virus-like particles were mixed with equal volume of 0% FBS complete media. The liposome-based transfection reagent was vortexed. 1/10 of the final volume of virus-like particle-media mixture of transfection reagent was added, left to complex for 15-20 minutes at 25° C., and added to wells in a dropwise manner. The wells were screened for fluorescence under confocal microscope imaging with TRITC, DAPI, GFP Cy4 filters set and brightfield.

To recover successfully transduced cells: the wells with greatest fluorescence were washed with PBS. The wells were trypsinized and the cell volume was transferred to a microcentrifuge tube. The tube was spun down for 5 minutes at 150 g at 25° C., the media was removed media and resuspended with PBS. The tube was spun down for 5 minutes at 150 g at 25° C., the media was removed media and resuspended with PBS a second time.

### Example 7B

### Liposome Optimization Protocol

Liposome complexation with JCV is optimized by varying the ratio of liposome to JCV and comparing the results to a number of controls.

Reagents included: Purified VLPs; 293 FT cells; DMEM, high glucose, pyruvate, 1-glutamine, non-essential amino acids; Fetal Bovine Serum; Trypsin; Geneticin 100×; DNAsel; 1× PBS; Altogen Biosystems HEK293 Transfection Reagent. Thermo TurboFect Transfection Reagent.

Well plates were seeded with HEK or U2OS cells and grown in 5% FBS complete media in a 37° C. incubated under 5% CO₂ until 50% confluence for at least 24 hours.

The VLPs were purified using a 0.2 μm sterile filter and diluted 5× in 0% FBS complete media and then treated with 1 unit of DNAsel per 2 μg DNA. The mixture was incubated overnight at 25° C.

The liposome-based transfection reagent was vortexed. 1/20 of the final volume of VLP-media mixture of liposome transfection reagent was added and left to complex for 3 hours at 25° C. The ratio of liposome to VLP was varied for the optimization trial.

After the DNAsel treatment, the purified, complexed VLPs having 500 ng DNA (protected by the VLPs) were added to each well in a 24 well plate in a dropwise fashion.

For the negative control, a comparable amount of purified plasmid was added to 0% FBS complete media as above. 1 united of DNAsel per 2 μg of DNA was added and the mixture was left to incubate overnight at 25° C. The mixture was treated with liposome transfection reagent as in the steps above.

For a DNA-Liposome treatment control, 500 ng of DNA was added to 100 μL 0% FBS complete media for each well in a 24 well plate. The mixture was treated with liposome transfection agent as in the steps above.

For a positive control, a purified plasmid was complexed with TurboFect Transfection reagent (ThermoFisher Scientific) according to manufacturer directions and was then added to the cells.

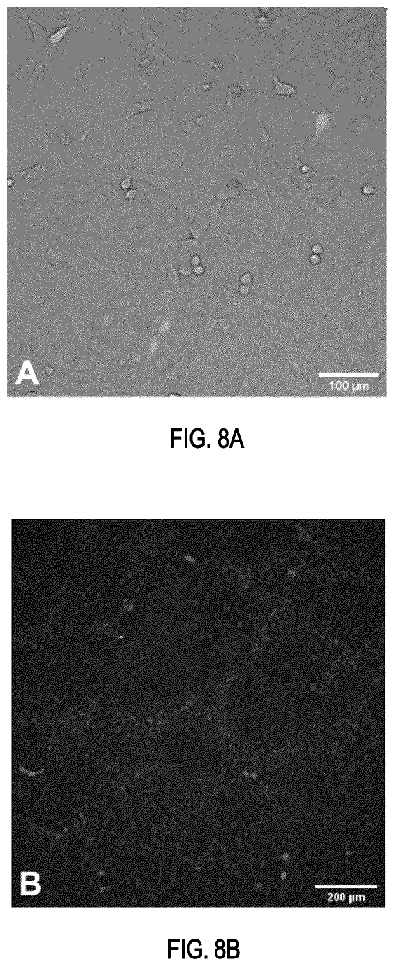

FIG. 8A shows a confocal microscope image using a 10× objective and a TRITC filter set with brightfield of U2OS cells transduced with unencapsulated JCV virus-like particles of the present disclosure. Brightfield was included to show nuclear localization of expressed mCherry.

FIG. 8B shows a confocal microscope image using a 10× objective and a TRITC filter of 293FT cells transduced with unencapsulated JCV virus-like particles of the present disclosure.

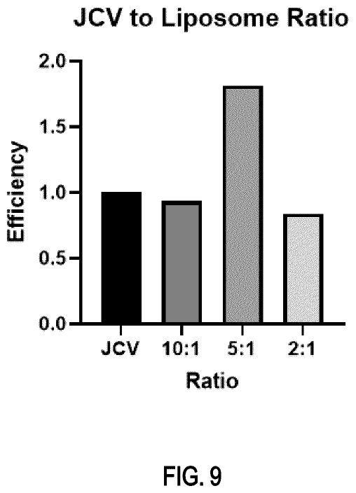

FIG. 9 shows a graph of transduction efficiency vs volume ratio for JCV virus-like particles of the present disclosure. All values were normalized to raw JCY efficiency (number of transduced cells/total number of cells), wherein n=1.

### Example 7C

### Mammalian Cell Transduction, Liposome Encapsulation, and Screening-Screening via FACS

In an alternative to selection at the level of well plates, the cells were pooled and individual cells that have successfully transduced were separated.

Well plates were seeded with HEK cells, which were grown in 5% FBS complete media in a 37° C. incubator under 5% CO₂ until 50% confluence (at least 24 hours). The virus-like particles were sterile filter purified with a 0.2 μm filter and then treated with DNAsel and incubated for 30 minutes at 25° C. Up to 1/20 of final media volume of purified virus-like particles was added to the wells along with 5 μL of DNAsel. The wells were left to transduce at 37° C. for 48 hours.

For liposome coated virus-like particles: Purified, filtered and DNAsel treated virus-like particles were mixed with equal volume of 0% FBS complete media. The liposome-based transfection reagent was vortexed. 1/10 of the final volume of virus-like particle-media mixture of transfection reagent was added, left to complex for 15-20 minutes at 25° C., and added to wells in a dropwise manner. In the dark, the wells were washed gently with PBS. The wells were typsinized and the cell volume was transferred to a microcentrifuge tube and spun down for 5 minutes at 150 g at 25° C. The media was removed and resuspended with sorting buffer in a microcentrifuge tube. The microcentrifuge tube was spun for 5 minutes at 150 g at 25° C., and the media was removed and resuspended with sorting buffer in a microcentrifuge tube a second and a third time. After final resuspension, the solution was filtered through a 70 μm filter. The solution was immediately loaded into a flow cytometer and sorted based on fluorescence. The cells with fluorescence were collect for nucleic acid extraction.

### Example 8

### Nucleic Acid Extraction

After finding a successful phenotype, it was correlated to the genotype, which involves extraction of the nucleic acid contained within the cells. The procedure of chloroform extraction is applicable to both bacterial and mammalian cells, as well as proteins.

Reagents included: Purified VLPs; HEK 293FT cells; Pelleted E. coli cells; Chloroform:Phenol:Iosamyl Alcohol (25:24:1); Chloroform; 100% Ethanol; 70% ethanol; Sodium Chloride; Agarose gels; Zymo Gel extraction kit; Deionized water.

In a microcentrifuge tube, the same volume of Chloroform: Phenol: Isoamyl Alcohol was added as the volume of the sample. The sample was vortexed until an emulsion formed. The mixture was centrifuged at 80% of maximum speed for 1 minute at room temperature. The aqueous phase was transferred to a new tube. Depending on whether RNA or DNA is extracted, the organic phase may be used. The previous steps were repeated three times.

An equal volume of chloroform was added. The sample was vortexed until an emulsion formed. The mixture was centrifuged at 80% of maximum speed for 1 minute at room temperature.

The aqueous phase was transferred to a new tube. Depending on whether RNA or DNA is extracted, the organic phase may be used.

The volume sample was estimated and adjusted to 0.2 M NaCl. 2 volumes of ice-cold 100% ethanol was added and the sample was placed in ice bath for 2 hours. The DNA was recovered by centrifugation at 0° C. The contents of the tube was poured off and left to air dry at room temperature for 1 hour. The pellet was dissolved with 20 μL distilled water.

### Example 9

### Nucleic Acid Analysis

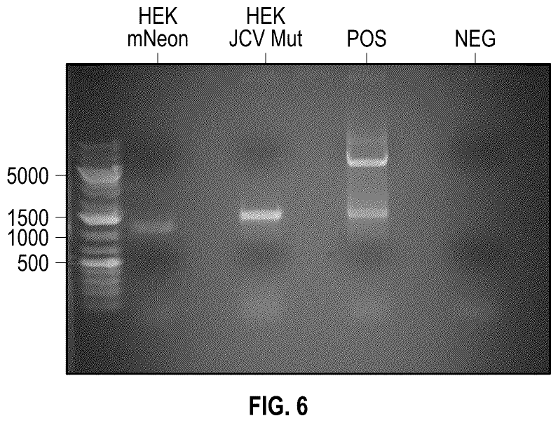

The extracted nucleic were synthesized via PCR before the next round of mutagenesis. An optional gel extraction protocol was used to validate the correct size as well as to increase purity. An example validation experiment can be seen in FIG. 6.

Reagents included: Template nucleic acids; Phire PCR master mix; Superscript RT-PCR master mix; agarose gels; Zymo gel extraction kit; Mutagenesis forward and backwards Gibson primers (for JCV, PEG10 or ARC); Random hexamers; DNA loading dye.

If dealing with RNA, RT-PCR was performed as described in the Superscript protocol (ThermoFisher Scientific). The RNA was primed with random hexamers.

Phire PCR was performed with Gibson primers, 20 μL reaction. The sample was nanodropped to determine DNA concentration. The sample was mixed with 4 μL DNA loading dye and loaded into a gel. Gel electrophoresis was performed to confirm band size. The band was excised, and a gel extraction was performed according to the Zymo gel extraction protocol. The DNA was stored in a −20° C. fridge. Some samples may be sequenced to track mutagenesis over iterative cycles. The Gibson mutagenesis primers may double as sequencing primers.

### Example 10

### In Vivo Screening in Mice

In order to test effective delivery of optimized virus-like particles, liposome-complexed virus-like particles carrying a reporter, such as GFP, is injected intravenously at the tail vein every other day. Injections subcutaneously or into the cerebrospinal fluid are also possible. In order to screen for specific delivery, the brain, heart, lung, liver, kidney, bladder, and spleen are removed, sectioned, and assessed for successful gene transduction by measuring fluorescence of the reporter tag (green fluorescence for GFP). Additionally, uncomplexed virus-like particles are injected directly into the cerebrospinal fluid and assessed for transduction in the brain. The dual expression plasmid allows mutants concentrated in a specific organ to be recovered via PCR. In order to test transduction efficiency, uncomplexed JCV virus-like particles are injected subcutaneously in immunodeficient nude mice with human adenocarcinoma cells. After 7 days post inoculation, optimized virus-like particles of the present disclosure with either GFP or luciferase encoding DNA are administered intravenously via tail vein injections. The tumor nodules are then removed, sectioned, and assessed for fluorescence or luminescence. Since mice do not have a natural immune response to JCV, it is possible to assess the immunogenicity of the virus-like particles through a humanized mouse model. The mouse health and survival are assessed after injecting both uncomplexed and liposome-complexed virus-like particles. Negative controls include PBS and cotreatment with an antiviral such as Ganciclovir.

### Example 11

### Organotypic Brain Slice Culture Treatment

To show effective gene delivery to mouse neurons and oligodendrocytes, organotypic mouse brain slices were used. This demonstrated the usefulness of liposome complexation to VLPs of the present disclosure to allow for delivery of a cargo to mouse tissue, which is normally restricted to human cells for JCV deliver, allowing for the characterization wherein the gene delivery vehicles of the present disclosure are able to deliver cargo within brain tissue.

Reagents included: Mice, Isoflurane, ACSF (see below), Slice Media (see below), Purified VLPs, Altogen Biosystems In Vivo Lipo Transfection Reagent, DNAsel, DMEM, 4% Paraformaldehyde in PBS 7.4 pH, 1× PBS, sodium azide.

A 6-well plate was prepared with inserts by transferring 750 μL of slide media to each well below the membrane inserts and pre-warming the plate in a 37° C. incubate.

Slide media included: Slice media: 66.7% v/v MEM w/Hank's Balanced Salt Solution, 4 mM Glutamine, 25 mM HEPES, 35 mM Glucose, 18 mM NaHCO₃, 25% v/v heat inactivated horse serum, 100 U/mL penicillin/streptomycin, 1× B-27 Plus Supplement (Gibco), Amphotericin B 2.5 ug/mL, 7.2 pH, filter sterilized through 0.1 μm sterile filter.

Mice (P25-30) which were singly-house were anesthetized with isoflurane before tissue collection. The mice were perfused wit ice-cold sterile filtered artificial cerebrospinal fluid (ACSF) containing: 119 mM NaCl, 2.5 mM KCl, 1 mM NaH2PO4, 26.2 mM NaHCo3, 11 mM glucose, 1.3 mM MgSO4 and 2.5 mMCaCl2.

The brain was decapitated, removed, and sectioned via 150 um coronal slices on a vibratome in ice cold ACSF. Once sectioned, the slices were transferred to a plate with ice cold slide media and transferred into a biosafety cabinet. In the biosafety cabinet, using a cut p1000 pipette tip, brain slices were transferred to the pre-warmed membrane inserts, and excess media was removed from the top of the inserts. The culture plate was transferred into a 37° C. incubator with 5% CO₂, and the slices were left to attach to the membrane for at least 48 hours. The media under the inserts was replaced once per week.

For treatment with VLPs and liposome, the VLPs were sterile filtered with a 0.2 μm filter and diluted 5× in DMEM. The VLPs were then treated with 1 unit of DNAseI per μg DNA, incubating overnight at 25° C. The liposome-based transfection reagent was vortexed. 1/50 of the final volume of VLP-media mixture of in vivo liposome transfection reagent was added and left to complex for 3 hours at 25° C. 750 μL of VLP-media mixture was added to the top of the membranes and incubated in a 37° C. incubator with 5% CO₂ for 1 hour. The excess media was removed from the top of the membranes, and the membranes were left to express for at least 24 hours before fixation and imaging.

For fixation, the plate was removed from the incubator, and the inserts were transferred to a new 6 well plate containing 1.5 mL of PBS in each well. The plate was left to shake at room temperature for 3 minutes. The wash was repeated two more times. The inserts were transferred into well plates with 1.5 mL of 4% PFA in PBS and incubated at 4° C. for 3 hours. The wells were washed in PBS three more times in a new 6 well plate and stored in PBS with 0.02% w/v sodium azide at 4° C. until imaging.

1. A method of producing virus-like particles in bacteria comprising:
mutating a DNA sequence of a gene of interest;
synthesizing the mutated DNA sequence with a plasmid, wherein a generated plasmid is formed;
transforming the generated plasmid into bacteria;
producing a protein of interest via a method of inducing gene expression from at least one transformed bacterial cell;
lysing a volume of the at least one transformed bacterial cell;
separating the protein of interest from cellular debris;
collecting the virus-like particles;
purifying the virus-like particles, wherein the virus-like particles comprise a structure in which at least two proteins are present in an aggregated form, and wherein the at least two proteins enclose a cavity; and
mixing the virus-like particles with liposomes, wherein the virus-like particles are complexed with the liposomes.

2. The method of claim 1, wherein the complexed virus-like particles have a higher transduction efficiency compared to un-enveloped virus-like particles.

3. The method of claim 1, wherein the plasmid size is increased, and wherein the virus-like particles have an increased packaging capacity of at least 5 kbp DNA.

4. The method of claim 1, wherein the plasmid size is increased, and wherein the virus-like particles have an increased packaging capacity of at least 7 kbp DNA.

5. The method of claim 1, wherein the plasmid size is increased, and wherein the virus-like particles have an increased packaging capacity of at least 9.4 kbp DNA.

6. The method of claim 1, wherein purifying the virus-like particles comprises using multimodal resin.

7. The method of claim 1, wherein the method is repeated at least a second time until a selected virus-like particle phenotype(s) is obtained.

8. The method of claim 1, wherein the method is repeated to select for a phenotype(s).

9. The method of claim 1, wherein the method of inducing gene expression from at least one transformed bacterial cell comprises arabinose induction.

10. The method of claim 1, wherein the bacteria is Escherichia coli.

11. The method of claim 1, further comprising a method of directed evolution, wherein the method of directed evolution comprises expressing a mutant DNA library in bacteria; screening for successful gene delivery; and recovering mutant genes.

12. A dual expression plasmid, wherein the dual expression plasmid is capable of producing at least one virus-like particle, and wherein the dual expression plasmid is selected for at least one phenotype in a mammalian cell.

13. The dual expression plasmid of claim 12, wherein the dual expression plasmid is transformed into Escherichia coli.

14. The dual expression plasmid of claim 12, wherein the dual expression plasmid comprises an expression cassette for major capsid protein 1 (VP1) of the Jon Cunningham Virus (JCV or human polyomavirus 2).

15. The dual expression plasmid of claim 12, wherein the plasmid produces a virus-like particle having a packaging capacity of at least 5 kbp DNA.

16. A method of producing virus-like particles in bacteria comprising:
mutating a DNA sequence of a gene of interest;
synthesizing a mutated sequence with a plasmid, wherein a generated plasmid is formed;
transforming the generated plasmid into a bacteria cell;
producing a protein of interest via a method of inducing gene expression from at least one transformed bacterial cell;
lysing a volume of the at least one transformed bacterial cell;
separating the protein of interest from cellular debris;
collecting the virus-like particles; and
purifying the virus-like particles, wherein the virus-like particles comprise a structure in which at least two proteins are present in an aggregated form, and wherein the at least two proteins enclose a cavity.

17. The method of claim 16, wherein the plasmid size is increased, and wherein the virus-like particles have an increased packaging capacity of at least 5 kbp DNA.

18. (canceled)

19. (canceled)

20. The method of claim 16, wherein purifying the virus-like particles comprises using multimodal resin.

21. The method of claim 16, wherein the method is repeated at least a second time until a selected virus-like particle phenotype(s) is obtained.

22. The method of claim 16, wherein the method is repeated to select for a trait(s).

23. The method of claim 16, wherein the method of inducing gene expression from at least one transformed bacterial cell comprises arabinose induction.

24. The method of claim 16, further comprising a method of directed evolution, wherein the method of directed evolution comprises expressing a mutant DNA library in bacteria; screening for successful gene delivery; and recovering mutant genes.

25. The method of claim 16, wherein the transduction efficiency of the virus-like particle is increased by a method comprising:
transducing a virus-like particle in a mammalian cell;
selecting the mammalian cell with a selected phenotype;
extracting at least one nucleic acid from the selected mammalian cell; and
purifying the at least one nucleic acid.

26. The method of claim 25, wherein the at least one nucleic acid is extracted by chloroform extraction.

27. The method of claim 25, further comprising optionally validating a size and a purity of the virus-like particle by gel electrophoresis.

28. The method of claim 25, wherein the at least one purified nucleic acid undergoes a round of mutagenesis until a selected phenotype and/or genotype is obtained.
